(19) United States
(12) Patent Application Publication
(10) Pub. No.: US 2011/0144577 A1
(43) Pub. Date: Jun. 16, 2011

(54) HYDROPHILIC COATINGS WITH TUNABLE COMPOSITION FOR DRUG COATED BALLOON

(76) Inventors: John Stankus, Campbell, CA (US); Stephen Pacetti, San Jose, CA (US); Mikael Trollsas, San Jose, CA (US); Syed Hossainy, Hayward, CA (US)

(21) Appl. No.: 12/636,158

(22) Filed: Dec. 11, 2009

(51) Int. Cl. A61M 29/00; A61K 47/32; A61K 31/44; A61P 9/10

(52) U.S. Cl. 604/96.01; 514/772.5; 514/772; 514/291

(57) ABSTRACT

A tunable coating formulation is described for a drug delivery balloon comprising a therapeutic agent, an excipient and a plasticizer. The tunable coating includes a first therapeutic agent and a first excipient, and can have a second therapeutic agent and a second excipient. The first and second therapeutic agents have different dissolution rates during balloon inflation and therefore provide a coating that is tunable. The plasticizer in the formulation has a weigh ratio of excipient to plasticizer below 1:0.1.

### FIELD OF THE INVENTION

The disclosed subject matter is related to the delivery of drugs from an insertable medical device. More particularly, the disclosed subject matter relates to a medical device including a balloon for delivery of a therapeutic agent, the balloon having a formulation with a tunable excipient.

### BACKGROUND OF THE INVENTION

Atherosclerosis is a syndrome affecting arterial blood vessels. It is a chronic inflammatory response in the walls of arteries, which is in large part due to the accumulation of lipid, macrophages, foam cells and the formation of plaque in the arterial wall. Atherosclerosis is commonly referred to as hardening of the arteries although the pathophysiology of the disease manifests itself with several different types of lesions ranging from fibrotic to lipid laden to calcific. Angioplasty is a vascular interventional technique involving mechanically widening an obstructed blood vessel, typically caused by atherosclerosis.

During angioplasty, a catheter having a tightly folded balloon is inserted into the vasculature of the patient and is passed to the narrowed location of the blood vessel at which point the balloon is inflated to a fixed size using an inflation fluid, typically angiographic contrast media. Percutaneous coronary intervention (PCI), commonly known as coronary angioplasty, is a therapeutic procedure to treat the stenotic coronary arteries of the heart, often found in coronary heart disease.

In contrast, peripheral angioplasty, commonly known as percutaneous transluminal angioplasty (PTA), refers to the use of mechanical widening of blood vessels other than the coronary arteries. PTA is most commonly used to treat narrowing of the arteries of the leg, especially, the iliac, external iliac, superficial femoral and popliteal arteries. PTA can also treat narrowing of veins and other blood vessels.

It was determined that following angioplasty, although a blood vessel would be successfully widened, sometimes the treated wall of the blood vessel experienced abrupt closure after balloon inflation or dilatation, due to acute recoil or spasm. Interventional cardiologists addressed this problem by stenting the blood vessel to prevent acute recoil and vasospasm. A stent is a device, typically a metal tube or scaffold, which was inserted into the blood vessel following angioplasty, in order to hold the blood vessel open.

While the advent of stents eliminated many of the complications of abrupt vessel closure after angioplasty procedures, within about six months of stenting, a re-narrowing of the blood vessel can form, which is a condition known as restenosis. Restenosis was discovered to be a response to the injury of the angioplasty procedure and is characterized by a growth of smooth muscle cells—analogous to a scar forming over an injury. As a solution, drug eluting stents were developed to address the reoccurrence of the narrowing of blood vessels. One example of a drug eluting stent is a metal stent that has been coated with a drug that is known to interfere with the process of restenosis. A potential drawback of certain drug eluting stents is known as late stent thrombosis, which is an event in which blood clots inside the stent.

Drug eluting balloons are believed to be a viable alternative to drug eluting stents in the treatment of atherosclerosis. In a study which evaluated restenosis, and the rate of major adverse cardiac events such as heart attack, bypass, repeat stenosis, or death in patients treated with drug eluting balloons and drug eluting stents, the patients treated with drug eluting balloons experienced only 3.7 percent restenosis and 4.8% MACE as compared to patients treated with drug eluting stents, in which restenosis was 20.8 percent and 22.0 percent MACE rate. (See, PEPCAD II study, Rotenburg, Germany).

Although drug eluting balloons are a viable alternative and in some cases may have greater efficacy than drug eluting stents as suggested by the PEPCAD II study, drug eluting balloons present challenges due to the very short period of contact between the drug coated balloon surface and the blood vessel wall. The drug delivery time period for a drug coated balloon differs from that of a controlled release drug eluting stent, which is typically weeks to months. In particular, the balloon can only be inflated for less than one minute, and is often inflated for only thirty seconds. Therefore, an efficacious, therapeutic amount of drug must be transferred to the vessel wall within a thirty-second to one-minute time period. For the peripheral vasculature, the allowable inflation times can be greater than one minute, but are still measured in minutes. Thus, there are challenges specific to drug delivery via a drug coated balloon because of the necessity of a short inflation time, and therefore time for drug or coating transfer—a challenge not presented by a drug eluting stent, which remains in the patient's vasculature once implanted.

Other considerations are the current theories about the mechanism by which a drug coated balloon transfers drug to the vessel wall. One theory, for example, is that upon balloon expansion, drug mechanically fractures or dissolves from the coating, diffuses to the vessel wall and then permeates into the vessel wall. A second theory is that upon balloon expansion the balloon coating is transferred to the vessel wall, and then drug permeates into the vessel wall from the coating adhered to the vessel wall. Another theory is that the balloon expansion creates tears and microfissures in the vessel wall and a portion of the coating inserts into the tears and microfissures. Drug then permeates into the vessel wall from the coating within the tears and fissures. Yet another theory is that upon balloon expansion, a layer of dissolved drug and coating excipients is formed at a high concentration on the vessel wall as a boundary layer. The drug diffuses and permeates from this boundary layer into the vessel wall. In most of these theories, the drug transfers from the balloon to the circulation or the vascular wall tissue upon fracture of the coating due to inflation of the balloon and occurs within one minute, and preferably within 30 seconds. Therefore, a need exists for a drug coated balloon having efficient drug transfer to a vessel wall.

Various embodiments of drug-coated balloons have been proposed, including balloons with a therapeutic agent disposed directly on the balloon surface and balloons having various protective sheaths. However, not all embodiments result in an efficacious response in reducing restenosis after balloon and bare metal stent trauma.

Therefore, a need exists for a drug delivery balloon, and more particularly, a balloon coated with a therapeutic agent that provides for effective delivery kinetics of the therapeutic agent from the surface of the balloon.

### SUMMARY OF INVENTION

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

In accordance with an aspect of the disclosed subject matter, coating formulation for a medical balloon is provided. The coating includes a cytostatic therapeutic agent, an excipient, and a plasticizer. The excipient to plasticizer has a weight ratio ranging from 1:20 to 20:1 or more preferably from 10:1 to 1:10, and most preferably from 5:1 to 1:1.

It has been found that the coating of the disclosed subject matter when applied to a medical balloon exhibits improved flexibility, and decreased brittleness. A coating with improved flexibility and decreased brittleness is important for the coating to withstand the balloon post-coating processes such as balloon pleating, folding, sheathing and packaging that occur in the dry state. For a consistent product, is it important for the balloons to have good dose control. It is also important for the balloons to be folded and pressed down to a small profile to facilitate delivery to the lesion site. Consequently, if the balloon coating were brittle, it could be shed during balloon folding and pressing operations. This can lead to variability in the drug dose. It can also lead to drug contamination of manufacturing equipment.

In one embodiment, the excipient is poly(vinyl pyrrolidone) (PVP) and the plasticizer is glycerol. In another embodiment, the cytostatic therapeutic agent is zotarolimus. Preferably, the cytostatic therapeutic agent and excipient have a weight ratio of greater than 1:1. It has been determined that such ratios lead to improved drug recovery when tracking the medical balloon to a lesion site before inflation of the balloon. In another embodiment, the glass transition temperature of the coating is below ambient temperature.

The coating of the disclosed subject matter provides advantages such as minimized drug loss during folding of the balloon, improved drug recovery upon tracking the balloon through a lumen of a subject. The coating can be applied to the balloon, in particular, an outer surface of the balloon by a variety of methods. One such method is direct coating techniques.

It is to be understood that both the foregoing description is exemplary and is intended to provide further explanation of the disclosed subject matter claimed to a person of ordinary skill in the art. The accompanying drawings are included to illustrate various embodiments of the disclosed subject matter to provide a further understanding of the disclosed subject matter. The exemplified embodiments of the disclosed subject matter are not intended to limit the scope of the claims.

### BRIEF DESCRIPTION OF DRAWINGS

The disclosed subject matter will now be described in conjunction with the accompanying drawings in which:

FIG. 1A is a planar view of one representative balloon catheter in accordance with the disclosed subject matter; and FIG. 1B is a cross-sectional view taken along lines A-A in FIG. 1A in accordance with one embodiment of the disclosed subject matter.

FIG. 2 is a graph illustrating percent drug release as a function of drug and plasticizer content in the formulation in accordance with one embodiment of the disclosed subject matter.

FIG. 3 are images demonstrating the effect of adding a plasticizer to a PVP-C30 balloon coating including without (left panel) and with (right panel) twenty percent glycerol by weight.

FIG. 4 is a graph illustrating comparative mean drug recoveries as a function of as coated (AS) or folded pressed sheathed (FPS) coated balloons of two formulations in accordance with one embodiment of the disclosed subject matter.

FIG. 5 contains images demonstrating the effect of changing coating process conditions on the resulting coating morphology including coating from 95:5 acetone:ethanol (left panel) or 85:15 acetone:ethanol (right panel) by weight.

### DETAILED DESCRIPTION

Reference will now be made in detail to the various aspects of the disclosed subject matter. The method of the disclosed subject matter will be described in conjunction with the detailed description of the device, the figures and examples provided herein.

The devices and methods presented can be used for delivery within and/or treating of the lumen of a patient. In particular, the disclosed subject matter is particularly suited for treatment of the cardiovascular system of a patient, such as performance of angioplasty and delivery of a balloon expandable medical device, such as a stent, filter and coil.

In accordance with the disclosed subject matter, a balloon catheter is provided for delivery of a therapeutic agent, the balloon including an outer surface having a tunable coating disposed on at least a length of the outer surface. The tunable coating includes a therapeutic agent and an excipient. The solubility of the coating in vivo,the biosolubility, is tunable based on the substances and concentrations chosen for the therapeutic agent and excipient.

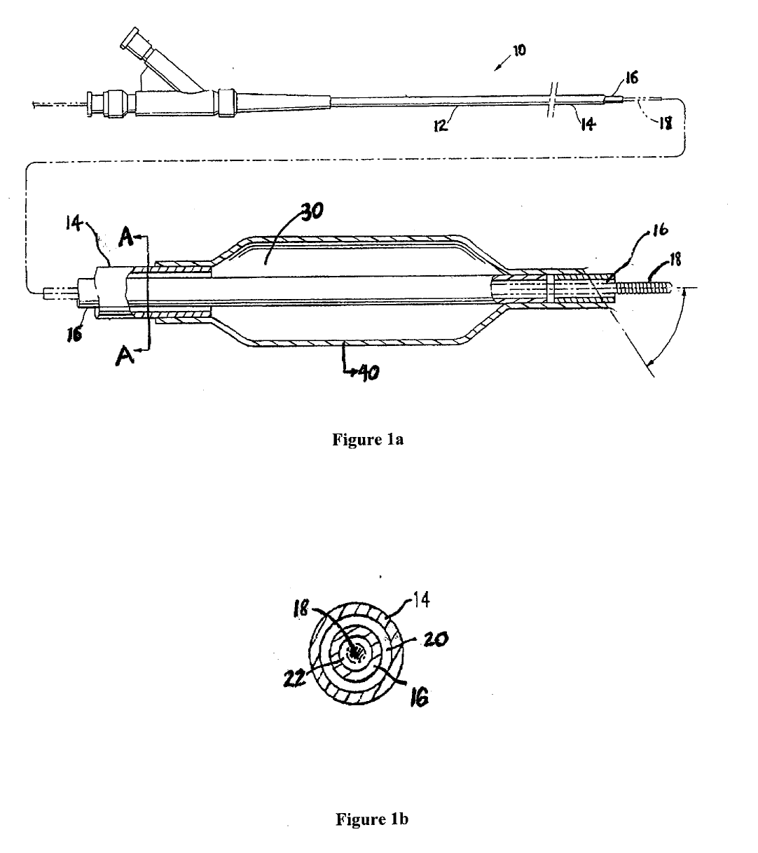

Referring to FIG. 1, for purposes of illustration and not limitation, an exemplary embodiment of balloon catheter device in accordance with the disclosed subject matter is shown schematically in FIGS. 1A and 1B. As depicted in FIGS. 1A and 1B, the balloon catheter device 10 generally includes an elongated catheter shaft 12 having a proximal end and having a distal end and an expandable balloon 30 located proximate to the distal end of the catheter shaft. The expandable balloon has an outer surface and an inner surface disposed at the distal end portion of the catheter shaft. In accordance with the disclosed subject matter, a tunable coating 40 is applied to at least one length of the balloon catheter, the tunable coating including a first therapeutic agent and a first excipient, and can have a second therapeutic agent and a second excipient, wherein the first and second therapeutic agents have different dissolution rates during balloon inflation. In accordance with a preferred embodiment, as illustrated by way of example and not limitation in FIG. 1A, the coating is applied to at least one length of the outer surface of the balloon catheter.

The elongated catheter shaft 12 comprises an outer tubular member 14 and an inner tubular member 16. The outer tubular member 14 defines an inflation lumen 20 that can be disposed between the proximal end portion and the distal end portion of the catheter shaft 12. Specifically, as illustrated in FIG. 1B, the coaxial relationship between the inner tubular member 16 and the outer tubular member 14 defines an annular inflation lumen 20. The expandable member 30 is placed in fluid communication with the inflation lumen 20. The inflation lumen can supply fluid under pressure, and establish negative pressure to the expandable member. The expandable member 30 can thus be inflated and deflated. The elongated catheter is sized and configured for delivery through a tortuous anatomy, and can further include a guidewire lumen 22 that permits it to be delivered over a guidewire 18. As illustrated in FIG. 1b, the inner tubular member 16 defines the guidewire lumen 22 for the guidewire 18. Although FIGS. 1A and 1B illustrate the guidewire lumen as having an over-the-wire (OTW) construction, the guidewire lumen can be configured as a rapid-exchange (RX) construction, as is well known in the art.

In accordance with the disclosed subject matter, the coating is tunable with respect to its solubility. Therefore, the drug delivery balloon is able to provide the desired delivery kinetics as a result of its tunability. The choice of excipient is key in determining efficacy factors such as, retaining of the therapeutic agent during delivery, releasing of the therapeutic agent during deployment, minimizing systemic dosing, maximizing agent delivery efficiency and therapeutic effect, and preventing particulate generation and related thromboses, among other factors.

As used in accordance with the disclosed subject matter, “tunable” refers to the ability to be tuned or adjusted for desired functioning. Accordingly, a tunable coating refers to a coating that can be adjusted according to various parameter discussed herein.

In accordance with the disclosed subject matter, the balloon includes a tunable coating that comprises a therapeutic agent and an excipient. As disclosed herein, the tunable coating includes a first therapeutic agent and a first excipient and a second therapeutic agent and a second excipient. The coating has a biosolubility that is tunable based on the substances and concentrations chosen for each of the therapeutic agent and excipient. Preferably, the therapeutic agents have different dissolution rates. The coating can include additional therapeutic agents and excipients.

In accordance with the disclosed subject matter, the solubility of the coating can be adjusted by modifying a number of factors, including excipient type, composition and molecular weight of the excipient, modulation of excipient or polymer properties such as aqueous solubility, octanol/water partition coefficient, HLB (hydrophile-lipophile balance) number, glass transition temperature, degree of amorphous versus crystalline polymer, and orientation. Furthermore, the solubility or dissolution rates of the coating can be adjusted by varying the therapeutic agent concentration, therapeutic agent to excipient ratio, or coating thickness. Accordingly, these factors can be varied in order to provide a coating with the desired solubility and drug delivery kinetics.

The tunable coating provides for dissolution rates during balloon inflation that can be characterized generally as ranging from fast, soluble, intermediate, slow, extra slow, and non-soluble. Depending on the target tissue or vasculature where the therapeutic agent is to be delivered, the coating can be tuned such that the dissolution rate provides for effective drug delivery and uptake. A “fast” coating dissolution rate will typically have a dissolution time of less than 1 minute. A “soluble” coating dissolution rate will typically have a dissolution time ranging from about 1 minute to about 1 hour. An “intermediate” coating dissolution rate will typically have a dissolution time ranging from about 1 hour to about 2 weeks. A “slow” coating dissolution rate will typically have a dissolution time ranging from about 2 weeks to about 3 months. An “extra slow” coating dissolution rate will typically have a dissolution time ranging from about 3 months to 2 years. A “non-soluble” coating dissolution rate will typically have a dissolution time greater than 2 years. However, it shall be noted that the specific dissolution of a coating composition is dependent upon an interplay between input factors and that the dissolution rates provided herein are, therefore, recited as ranges.

The excipients include various oil-based soluble, water soluble, biosoluble, and durable or biodurable substances that are suitable for the delivery of a therapeutic agent. Biosolubility indicates solubility in a relevant biological media, such as blood. A substance which is not intended to degrade in the body, or which degrades only very slowly, is biodurable.

In accordance with a preferred embodiment, the excipients of the disclosed subject matter are water soluble. The excipients can include non-ionic hydrophilic polymers. Non-ionic hydrophilic polymers include, but are not limited to, poly(vinyl pyrrolidone) (PVP, povidone), silk-elastin like polymer, poly(vinyl alcohol), poly(ethylene glycol) (PEG), pluronics (PEO-PPO-PEO), poly(vinyl acetate), poly(ethylene oxide) (PEO), PVP-vinyl acetate (copovidone), polysorbate 80 (Tween 80) and polysorbate 20 (Tween 20). Preferably, the molecular weight of non-ionic hydrophilic polymers can be less than 50 kDa for fast solubility. The excipient can also include fatty acids, fatty acid esters and triglycerides. Another category are peglylated phospholipids such as distearoylphosphatidylethanolaminepoly(ethylene glycol) 2000 (PEG-PE). Further, the excipient can be a lubricious material which improves spreading and uniformity of coating.

As disclosed herein, the excipient consists of a biocompatible plasticizer. Alternatively, the plasticizer can be added to the excipient to keep it soft and pliable. Plasticizers can allow for greater coating flexibility and elongation to prevent coating cracking during inflation or brittleness. Plasticizers include, but are not limited to, glycerol, ethanol, dimethylsulfoxide, ethyl lactate, benzyl alcohol, benzyl benzoate, Cremophor EL, Vitamin E, tocopherol, liquid PEG (MW<1000), triethyl citrate, tributyl citrate, acetyl tributyl citrate, acetyl triethyl citrate, dibutyl phthalate, dibutyl sebacate, dimethyl phthalate, triacetin, propylene glycol, glycerin, 2-pyrridone, and combinations thereof. Preferably, a biocompatible plasticizer is used.

In accordance with yet another embodiment, sugars, polysaccharides or cellulosics, can be used as binders for the particles. Polysaccharides include, but are not limited to, dextran, sulfonated dextran, hydrogenated dextran, chondroitin sulfate, sodium hyaluronate, hyaluronic acid, hyaluronan, chitosan, sodium alginate, sucrose, pectin, mannitol, carboxymethyl cellulose (CMC) sodium, methyl cellulose, ethyl cellulose, hydroxyethyl cellulose, hydroxypropyl cellulose, and hydroxypropylmethylcellulose. Certain negative charged polysaccharides will provide a mucoadhesive effect to enhance tissue drug retention. Furthermore, sugars such as mannitol will provide a decreased hygroscopic effect when blended with more moisture-sensitive active ingredients such as cytostatic drugs or moisture sensitive excipients. Water soluble cellulosic materials can enhance coating strength or brittleness.

In accordance with yet another embodiment, anti-coagulants can be used as an excipient. For example, heparin based polysaccharides can provide a minimally thrombogenic surface to prevent blood clotting on the balloon surface or minimize platelet activation induced by the procedure. Heparin based polysaccharides include, but are not limited to, heparin, heparin sulfate, heparin disaccharides, heparin fraction 1, heparin fraction 2, low molecular weight heparin, heparin ammonium, heparin calcium, heparin lithium, heparin lithium, and heparin zinc lithium. Low molecular weight heparin includes centaxarin, periodate-oxidized heparin, heparin sodium end-amidated, heparin sodium, and nitrous acid delaminated.

In accordance with a preferred embodiment of the disclosed subject matter, the excipient possesses a mucoadhesive property. This mucoadhesive property of the binder will lead to longer drug retention within the coating adhered to the vessel wall. In particular, negatively charged excipients such as some polysaccharides (e.g. sodium hyaluronate, sodium alginate) and some non-ionic hydrophilic polymers exhibit mucoadhesive properties. Any above carboxylated materials can also be lightly activated with esters such as nitrophenolate or NHS-esters (N-hydroxy succinimide) for increased mucoadhesiveness. Alternatively, any above materials can be lightly thiolated for increased mucoadhesiveness and continued solubility.

Additionally or alternatively, the excipient is or includes a contrast agent, including but not limited to, Iopromide (Ultravist), Omnipaque (Iohexol), Ioxaglate (Hexabrix), Ioversol (Optiray), Iopamidol (Isovue), Diatrixoate (Conray), Iodixanol (Visipaque), and Iotrolan. At an intermediate coating thickness, a lower molecular weight (<1 kDa) hydrophilic contrast agent such as Iopromide (Ultravist) would enable faster therapeutic release and a slightly higher viscous coating of the vessel wall as compared with drug alone. The contrast agents are lipophilic and can aid in drug uptake and retention into the tissue wall. As disclosed herein, Ultravist and Optiray can be used given their more benign in vitro history of effects to smooth muscle and endothelial cells.

In accordance with yet another embodiment, excipients can consist of carboxylated aromatics similar in molecular structure to the structure used in contrast agents but without iodide substituents. These negatively charged carboxylated aromatic structures can be alkylated (C2-C 12) to optimize drug tissue uptake, or halogenated with fluoride, chloride or bromide for the same reason. The negatively charged structures are beneficial for tissue adhesiveness.

Table 1 provides non-limiting examples of the solubility enhancement provided by excipients that can be used in accordance with the disclosed subject matter:

TABLE 1
Solubility Enhancement of Zotarolimus
with Select Excipients in PBS

| Excipient Solution | Zotarolimus Solubility |
|---|---|
| (5% w/w in PBS) | (ug/ml, n = 3) |
| PVP C-17 | 5.6 ± 1.6 |
| Hydroxypropyl-β-cyclodextrin | 11.6 ± 3.1 |
| PEG 400 | 31.5 ± 3.5 |
| Glycerol | 43.2 ± 30.1 |
| 5% γ-Cyclodextrin | 55.3 ± 34.3 |
| Vitamin E TPGS | 512 ± 49.5 |
| Tween 20 | 732 ± 94.7 |
| PEG-PE | 1020 ± 417 |

As illustrated in Table 1, the excipients provide acceptable solubility for the cytostatic drug, zotarolimus. The excipients Vitamin E TPGS, Tween 20, and PEG-PE demonstrate the largest increase in zotarolimus solubility.

Table 2 provides non-limiting examples of coating dissolution rates during balloon inflation and representative excipient examples.

TABLE 2
Examples of Delivery Kinetics and Expected
Variable Ranges for Balloon Coatings

| Coating | | |
|---|---|---|
| Dissolution | | |
| Rate (during | Coating | |
| balloon | Dissolution | Representative Excipient |
| inflation) | Time | Example |
| Fast | <1 minute | Polyvinylpyrrolidone (PVP) |
| | | (MW <60 kDa) or Polyethylene glycol |
| | | (PEG) (lower MW <35 kDa) |
| Soluble | 1 min to 1 hour | Polyvinylpyrrolidone (PVP) |
| | | (MW >60 kDa) or Polyethylene oxide |
| | | (PEO) (higher MW >100 kDa) |
| Intermediate | 1 hour to 2 | Silk-elastin like protein polymers |
| | weeks | |
| Slow | 2 weeks-3 | Biodegradable polymer such as |
| | months | Poly(D,L-lactide-co-glycolide) |
| | | (PLGA) (50:50) |
| Extra Slow | 3 months-2 | Biodegradable polymer such as |
| | years | Poly(L-lactide-co-ε-caprolactone) |
| | | (PLLA:PCL) (70:30) |
| Non-Soluble | >2 years | Durable polymer such as |
| | | Poly(vinylidene fluoride-co- |
| | | hexafluoropropylene) |

As illustrated in Table 2 above, for a “fast” coating dissolution rate, representative excipient examples include, without limitation, poly(vinylpyrrolidone) (PVP) having a molecular weight less than about 60 kDa or polyethylene glycol (PEG) having a molecular weight less than about 35 kDa. The drug delivery mechanism and kinetics expected with this representative example include the release of the therapeutic agent with the coating during inflation. Further, the potential mucoadhesive polymer increases drug retention time on tissue or vasculature. Alternatively, or additionally, the lipophilic additive increases drug uptake in tissue.

As illustrated in Table 2 above, for a “soluble” coating dissolution rate, representative excipient examples include, without limitation, poly(vinylpyrrolidone) (PVP) having a molecular weight greater than about 60 kDa, or poly(ethylene glycol) (PEG) having a molecular weight greater than about 100 kDa. The drug delivery mechanism and kinetics expected with this representative example are similar to that of the “fast” coating dissolution rate, however, the slightly slower dissolution time allows for less drug wash off during balloon delivery before inflation.

As illustrated in Table 2 above, for an “intermediate” coating dissolution rate, representative excipient examples include, without limitation, silk-elastin like protein polymers. The drug delivery mechanism and kinetics expected with this representative example provides for enhanced systemic drug loss protection and absence of short-term solubility, therefore allowing for enhanced particulate safety. For an “intermediate” dissolution rate, the therapeutic agent is not released together with the coating but from the coating. The therapeutic agent release kinetics and transfer to tissue are significantly enhanced by mechanical action during balloon inflation. Typically, these type of coating materials can by hydrophilic and can swell to some extent upon hydration to aid in fast drug release.

As illustrated in Table 2 above, for a “slow” coating dissolution rate, representative excipient examples include, without limitation, biodegradable polymers such as Poly(D,L-lactide-co-glycolide) (PLGA) (50:50). The coatings from biodegradable hydrophobic polymers will offer enhanced systemic drug loss protection and particulate safety profile. The therapeutic agent is not released together with the coating but from the coating. Drug release kinetics and transfer to tissue are significantly enhanced by mechanical action during balloon inflation. Techniques such as using a thin coating, a polymer with a low glass transition temperature (Tg), and amorphous material or low crystalline material can provide for a more rapid drug release profile when using a biodegradable polymer.

As illustrated in Table 2 above, for an “extra slow” coating dissolution rate, representative excipient examples include, without limitation, biodegradable polymers such as poly(L-lactide-co-ε-caprolactone) (PLLA:PCL) (70:30). The drug delivery mechanism and kinetics are similar to a “slow” coating dissolution rate, however the degradation time is significantly extended. These coatings will have more long term degradation and mechanical stability under storage.

As illustrated above, for a “non-soluble” coating dissolution rate, representative excipient examples include, without limitation, durable polymers such as poly(vinylidene fluoride-co-hexafluoropropylene). The drug delivery mechanism and kinetics are similar to both a “slow” and “extra slow” coating dissolution rate, however the material is non-biodegradable. These non-soluble coatings will have the most chemical and mechanical stability under storage than other types.

In accordance with the disclosed subject matter, the outer surface of the balloon has a tunable coating that is disposed on at least a length of the outer surface. Preferably, the tunable coating includes a first therapeutic agent and a first excipient and a second therapeutic agent and a second excipient. In accordance with a preferred embodiment, the first and second therapeutic agents can have different dissolution rates during balloon inflation. Thus, the desired coating dissolution rates can be tunable and achieved as desired for either drug kinetics or safety profile. The delivery of the therapeutic agents can be modified and optimized to meet the therapeutic need. Furthermore, depending on the excipients used, the therapeutic agents can be released from the excipient or coating or with the excipient or coating. As disclosed herein, the first therapeutic agent is released from the coating, and the second therapeutic agent is released with the coating.

In one embodiment, the first therapeutic agent is different than the second therapeutic agent. Alternatively, however, the therapeutic agents can be the same.

In accordance with another embodiment, the coating can also include a third therapeutic agent and a third excipient. The therapeutic agents and excipients can be applied simultaneously to the balloon surface or they can be applied separately.

In accordance with yet another embodiment, the disclosed subject matter includes a balloon having a the tunable coating including a cytostatic drug and at least one excipient, wherein in the coating at least one polymeric component has a polydispersity index from about 1.05 to about 10, more preferably from 1.05 to 5. The polydispersity index (PDI), is a measure of the distribution of molecular mass in a given polymer sample. The PDI calculated is the weight average molecular weight divided by the number average molecular weight. It indicates the distribution of individual molecular masses in a batch of polymers. A smaller PDI value provides a more consistent dissolution rate among the polymeric excipient molecules.

It has been found that coatings can be tunable to achieve desirable dissolution and drug delivery kinetics. In this regard, the choice of an excipient or modified excipient can be important to define coatings exhibiting efficacy factors such as but not limited to: how the therapeutic agent is retained during delivery, how the agent is released during balloon inflation, minimizing systemic dosing, maximizing agent delivery efficiency and therapeutic effect, and preventing particulate loss, related thromboses and embolic events.

Accordingly, in one aspect of the disclosed subject matter, a method is provided for coating a medical device, such as a drug coated balloon. The coating includes a cytostatic therapeutic agent and an excipient having a tunable molecular architecture. As used herein the phrase “tunable molecular architecture” means selection of an appropriate excipient composition, molecular structure, functionality, and morphology including appropriate modifications to yield the desired coating dissolution and drug delivery kinetics. The molecular architecture can be tuned through the design of input variables such as monomer/polymer composition, aromaticity, hydrophilicity, molecular charge, neutrality, aliphatic chain length, density of functional groups, molecular weight, aqueous solubility, octanol/water partition coefficient, HLB number, glass transition temperature, and percent crystallinity. The method of the disclosed subject matter advantageously is capable of providing desired delivery kinetics as a result of its tunability.

In accordance with the disclosed subject matter, the method includes selecting a cytostatic therapeutic agent and an excipient, and blending or mixing the cytostatic agent and excipient to define a coating. The method can further include tuning the molecular structure of the coating such that specific characteristics of the coating that are important to product performance can be adjusted and optimized. Some of these characteristics include coating solubility, coating hydrophilicity, coating adhesion and cohesion, coating stability under sterilization and storage, drug release kinetics, drug solubility and stability, and safety profile including particulate hazard and re-endothelialization.

For example and not limitation, the molecular architecture of the excipient can be modified and tuned through the adjustment of several input parameters, as described in Table 3 below.

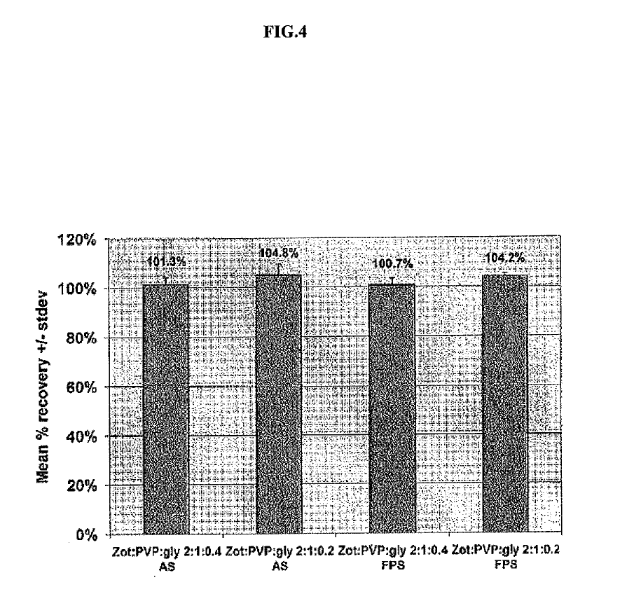

TABLE 3
Parameters that Affect Molecular Architecture and Hydrophilic
Excipient or Coating Characteristics

| Excipient | Effect(s) of | Effect(s) of | |
|---|---|---|---|
| Modification | Increase | Decrease | Example |
| Monomer/Polymer | Choice of excipient or coating | | Polyvinylpyrrolidone |
| Composition | composition has a large | | (PVP), glycerol, |
| | effect on all coating | | polyethylene glycol (PEG), |
| | characteristics listed above. | | polysorbate 20, polysorbate |
| | | | 60, polysorbate 80, benzyl |
| | | | benzoate, benzyl alcohol, |
| | | | dimethylsulfoxide, silk- |
| | | | elastin like protein polymer, |
| | | | propylene glycol, N-methyl |
| | | | pyrrolidone, soybean oil, |
| | | | dextran, albumin, |
| | | | carboxymethyl cellulose, |
| | | | ethanol, mannitol, pluronics |
| | | | F68, F127, PEG- |
| | | | phospholipids, iodinated |
| | | | polyfunctional benzoic acid |
| | | | and contrast agents.. |
| | | | Contrast agent for soluble |
| | | | coating agent. Thin |
| | | | poly(vinylidene fluoride-co- |
| | | | hexafluoropropylene) |
| | | | (PVDF-HFP) for durable |
| | | | coating. |
| Monomer/Polymer | A high interaction | More spacing | Increased L-lactide content |
| Composition- | between polymer | between chains can | in poly(L-lactide-co- |
| Intramolecular | chains can provide | provide more | caprolactone) copolymers |
| attraction: H- | for increased coating | amorphous content | can lead to higher |
| Bonding versus | mechanical stability, | and lower | crystallinity. Polyurethane |
| Steric Hindrance | cohesion, higher | crystallinity for | ureas exhibit increased H- |
| | crystallinity and | faster solubility and | bonding and mechanical |
| | greater molecular | decreased stability | stability. |
| | packing density. | of material. | |
| Hydrophilicity and | Will provide faster | Slower water | Non-ionic hydrophilic polymers |
| coating aqueous | water absorption, | absorption, slower | such as PVP and polyethylene |
| solubility | faster drug release, | drug release, and | glycol (PEG) are water soluble |
| | and faster coating | slower coating | and provide for fast coating |
| | dissolution. Time to | dissolution. Tmax | dissolution and drug delivery. |
| | maximum blood | would be increased. | Can increase or decrease PEG |
| | concentration | Particulate embolic | content as a soft segment in |
| | (Tmax) can be | hazard can be | polyurethanes to increase or |
| | decreased. | increased due to | decrease hydrophilicity. Non- |
| | | longer life of shed | ionic, hydrophobic polymers such |
| | | particles. | as PVDF-HFP and PCL are non- |
| | | | water soluble. |
| | | | The chi solubility parameter or |
| | | | cohesive parameter that describes |
| | | | the attractive strength between |
| | | | molecules of a material can be |
| | | | utilized to illustrate relevant |
| | | | properties for the DCB. Values for |
| | | | more hydrophobic durable (non- |
| | | | water soluble) polymers such as |
| | | | PVDF, PU, PUU and PDLLA |
| | | | range from 17-21 MPa^{(1/2)}. Chi |
| | | | solubility parameters for |
| | | | hydrophilic soluble polymers such |
| | | | as PVA and PVP are |
| | | | approximately 25-26 MPa^{(1/2)}. The |
| | | | solubility parameter for a lower |
| | | | molecular weight glycerol |
| | | | plasticizer is 33.8 MPa^{(1/2)}. Water |
| | | | has a value of 47.9 MPa^{(1/2)}. The |
| | | | solubility parameter for a mixture |
| | | | is defined as ${δ}_{mix} = {\sum }_{i} {δ}_{i} {φ}_{i}$ |
| | | | where φ₁ is the volume fraction of |
| | | | each component. Therefore, the |
| | | | closer the chi value of a material to |
| | | | that of water for the excipient |
| | | | mixture would be expected to |
| | | | result in greater aqueous solubility. |
| Molecular Charge | Charged molecules | Lower charge or | Charged polysaccharides |
| or Neutrality | tend to possess | more neutral species | such as sodium |
| | mucoadhesive | can exhibit less | carboxymethylcellulose, |
| | properties. | mucoadhesive | sodium hyaluronate, |
| | | properties. | chitosan, and sodium |
| | | | alginate can exhibit |
| | | | mucoadhesive properties as |
| | | | well as complex and/or |
| | | | physically bind negatively |
| | | | charged molecules. |
| Cyclic Chain | Cyclic chain will | Lower chain rigidity | Adjusting the ratio of cyclic |
| | increase chain | (lower Tg), faster | to aliphatic chain in |
| | rigidity (higher Tg), | release rate and | poly(ester amide) (PEA) |
| | therefore slow down | higher % | polymers. |
| | the release rate. The | elongation. | |
| | elasticity will also be | | |
| | reduced as a result. | | |
| Aliphatic Chain | Increased carbon | Decreased carbon | Adjusting length of |
| Length | chain lengths will | chain lengths will | hydrocarbon chains in |
| | lend towards | lend towards | Poly(ester amide) (PEA) |
| | increased flexibility | increased stiffness | polymers. |
| | (lower Tg) of | (higher Tg) of | |
| | material for highter | material for lower | |
| | % elongation and | % elongation and | |
| | slower drug release. | faster drug release. | |
| Density of | Will increase ability | Lower density of | RGD grafted low MW PEG |
| Functional Groups | to and density of | functional groups | can provide for increased |
| | grafted signaling or | and attached ligands | adhesion of released coating |
| | other molecules such | can allow for | to the vascular endothelial |
| | as RGD sequences | decreased steric | cell wall. |
| | for cell attachment. | hindrance and | |
| | | increased | |
| | | bioavailability. | |
| Molecular Weight | Higher molecular | Lower molecular | Lower molecular weight (<60 |
| | weight material will | weight could lead to | kDa). |
| | tend towards higher | decreased coating | poly(vinylpyrrolidone) is |
| | spray viscosity and | integrity (more | easily spray coated due to |
| | lower solubility in | cracks, brittleness) | its low spray viscosity and |
| | various spray | which translates to | high solubility in aqueous |
| | solvents and in | increased coating | and organic media as well. |
| | aqueous media. | loss during dry | Polyvinylpyrrolidone is |
| | Generally, a higher | balloon processing | quickly dissolved for fast |
| | molecular weight | and delivery prior to | drug release on inflation |
| | provide increased | balloon inflation. | from a drug coated balloon. |
| | mechanical strength | Lower molecular | A lower molecular weight |
| | and cohesion of a | weight provides for | (PVP C-15, 10,000 Mw) |
| | coating. This | improved clearance | can lead to increased |
| | increased cohesion | from the body. | zotarolimus loss on tracking |
| | could result in | Lower molecular | prior to inflation compared |
| | increased drug | weight in general | with PVP 30 of 60,000 Mw. |
| | recovery on the | results in faster | PVP C-17 is the highest |
| | balloon during | dissolution. | MW approved for parental |
| | tracking to the lesion | | application due to improved |
| | prior to balloon | | clearance of this and lower |
| | inflation. Higher | | MW PVP grades. |
| | molecular weight in | | |
| | general results in | | |
| | slower dissolution. | | |
| Glass Transition | Increased EtO | Decreased EtO | Certain low Tg poly(ε- |
| Temperature | sterilization stability | sterilization stability | caprolactone) based |
| | and slower drug | leading to coating | materials can provide a very |
| | release. Less | flow and faster drug | fast burst release of drug up |
| | flexible coating for | release. More | to 99% + release at 1 day. |
| | decreased handling | flexible coating for | Adding a plasticizer such as |
| | and catheter | enhanced catheter | glycerol to non-ionic |
| | processing | handling and | hydrophilic polymers such |
| | | processing. | as PVP can lower the dry |
| | | | coating Tg to increase |
| | | | coating flexibility. |
| % Crystallinity | Higher storage | Lower storage | Increasing or decreasing L- |
| | stability, slower drug | stability, faster | lactide content in poly(L- |
| | release, less flexible | solubility, faster | lactide-co-caprolactone) |
| | coating. | drug release, more | polyester copolymers. |
| | | flexible coating. | Adding HFP to PVDF-HFP |
| | | | copolymer to increase |
| | | | coating flexibility and drug |
| | | | release. |
| Drug to Excipient | A higher drug to | A low drug to | Coating based on |
| Ratio (D:E) | polymer ratio (>1:1) | polymer ratio (<1:1) | zotarolimus:PVP C- |
| | leads to improved | leads to improved | 17:glycerol of 2:1:0.4 (w/w) |
| | drug recovery on | solubilization of | gave improved drug |
| | tracking the device | hydrophobic drug as | recovery on tracking or |
| | to the lesion site | well as lower drug | delivery verses a ratio of |
| | before inflation. | recovery on | 1:1:0.4. |
| | | tracking the device | |
| | | to the lesion site | |
| | | before inflation. | |
| Excipient to | A larger excipient to | To minimize drug | Zot:PVP:glycerol coatings |
| Plasticizer Ratio | plasticizer ratio will | loss on dry folding a | of 2:1:0.4 or 2:1:0.2 are |
| (E:P) | result in a more | smaller excipient to | flexible enough to result in |
| | brittle coating, | plasticizer ratio is | minimal drug loss on dry |
| | permits more drug | used. However, too | folding, pleating and |
| | loss from the dry | small of an E:P ratio | sheathing of the drug coated |
| | coating on the | can result in a tacky, | balloon. If the E:P is < 1:0.1, |
| | balloon folding and | fluid coating. | then the coating can |
| | pressing during | | become more tacky and |
| | catheter preparation. | | fluid. An E:P > 1:0.1 can |
| | | | become more brittle. In he |
| | | | absence of plasticizer, the |
| | | | hydrophilic excipient can be |
| | | | quite brittle when dry. |
| Coating | A fast evaporation | A slower | With zotarolimus:PVP: |
| Processing | rate due to drying | evaporation rate due | glycerol 2:1:0.4 a clear |
| Condition | devices, higher | to absence of drying | or opaque coating can be |
| (evaporation rate) | percent solids, or | devices, lower | obtained as a function of the |
| | fast evaporating | percent solids, or | drying conditions (FIG 4). |
| | solvents during | use of slow | With fast evaporation |
| | coating can freeze in | evaporating solvents | using either an increased |
| | the morphology of | allows time for | acetone:ethanol ratio > 85:15 |
| | the coating and | coating components | or in-line drying a |
| | drug: excipient | to migrate, phase | clear morphology results. |
| | interactions. | separate, and | This clear, glassy |
| | | arrange the coating | morphology is more fragile |
| | | morphology more | and can result from |
| | | towards an | miscibility of the drug with |
| | | equilibrium state. | the excipients or drug |
| | | | complexation with PVP. |
| | | | With absence of in-line |
| | | | drying or using an |
| | | | acetone:ethanol ratio of |
| | | | 85:15 an opaque coating |
| | | | morphology is produced. |
| | | | This white coating appears |
| | | | to be more flexible and has |
| | | | superior dry coating |
| | | | integrity on a balloon. |
| Coating | Higher Crystallinity | Lower Crystallinity | High molecular weight PEG |
| Crystallinity | will slow coating | will tend to hasten | (MW > 2000) will crystallize, |
| | dissolution. Coating | the coating | dissolve more slowly and be |
| | brittleness and | dissolution. | more brittle. PEG 400 will |
| | hardness will be | Coating brittleness | not crystallize as it is a |
| | higher. | will be less and the | liquid and will make the |
| | | coating will be | coating softer and less |
| | | softer. | brittle. |
| Coating Tg when | A coating with a Tg | A coating with a Tg | A coating of zotarolimus |
| dry | above ambient | below ambient | and PVP C-17 only will |
| | temperature will be | temperature will be | have a Tg above room |
| | harder and more | softer but sticky | temperature. It will be hard |
| | brittle | | and brittle. A |
| | | | zotarolimus/PVP/glycerol |
| | | | formulation of 1:1:0.4 will |
| | | | have a Tg below room |
| | | | temperature. It is soft and |
| | | | tends to be sticky. |

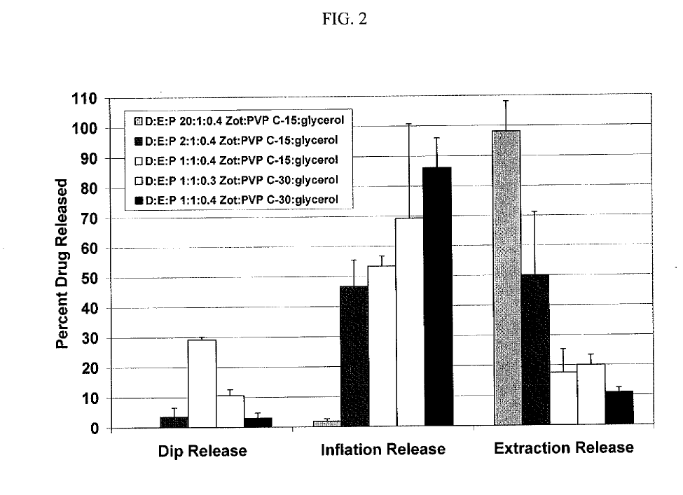

Referring to FIG. 2, the results from an in-vitro drug release test as a function of drug to excipient ratio is provided. This test is intended to mimic the drug released during the delivery of the balloon by dipping the balloon in release media, the balloon inflation in release media, and then how much is left on the balloon. As illustrated in FIG. 2, lowering the drug to excipient ratio to 1:1 for PVP C-15 caused an increased drug release on delivery or loss (or decreased drug recovery) on dipping as demonstrated. Such results indicate that a lower drug to excipient ratio results in lower drug recovery on tracking prior to inflation in-vivo.

Referring to FIG. 4, the mean drug recoveries as a function of coated (AS) or fold, pressed, and sheathed (FPS) with a coating formulation comprising zotarolimus:PVP:glycerol having a weight ratio of 2:1:0.4 is compared to the same coating formulation in a weight ratio of 2:1:0.2. The presence of glycerol in the coating at these levels maintains flexibility in the dry state and minimizes any loss from catheter processing such as folding, sheathing and packaging.

In accordance with the disclosed subject matter, the coating can be applied to the medical device by processes such as dip-coating, pipette coating, syringe coating, air assisted spraying, electrostatic spraying, piezoelectric spraying, spray drying, pneumatic spray, ultrasonic spray, spray with patterning, electrospinning, direct fluid application, or other means as known to those skilled in the art. The coating can be applied over at least a length or the entirety of the balloon or medical device. By way of example, and not limitation, certain coating processes that can be used with the instant disclosed subject matter are described in U.S. Pat. No. 6,669,980 to Hansen; U.S. Pat. No. 7,241,344 to Worsham; and U.S. Publication No. 20040234748 to Stenzel, the entire disclosures of which are hereby incorporated by reference. In accordance with one embodiment of the disclosed subject matter, the medical device is a balloon catheter and the coating can be applied to either a folded or inflated balloon. Furthermore, the coating can be directly applied into the folds of the folded balloons. The coating characteristics are affected by process variables. For example, for dip-coating process, coating quality and thickness can vary as an effect of variables such as number, rate, and depth of dips along with drying time and temperature.

In accordance with one embodiment, the balloon can be sprayed with therapeutic agent encapsulated in the durable excipient solution. Spray solvents can consist of the following class III solvents including but not limited to acetone, anisole, 1-butanol, 2-butanol, butyl acetate, tert-butylmethyl ether, cumene, dimethyl sulfoxide, ethanol, ethyl acetate, ethyl ether, ethyl formate, heptane, hexane, cyclohexane, isobutyl acetate, isopropyl acetate, methyl acetate, 3-methyl-1-butanol, methylethyl ketone, methylisobutyl ketone,cyclohexanone, 2-methyl-1-propanol, pentanel, 1-pentanol, 1-propanol, and propyl acetate, or blends thereof.

Additional spray solvents that can be used or blended with class III solvents include class H spray solvents. The class H spray solvents include but are not limited to, acetonitrile, chloroform, 1,2-dichloroethane, dichloromethane, 1,2-dimethyloxyethene, N,N-dimethylacetamide, N,N-dimethylformamide, 1,4-dioxane, 2-ethoxyethanol, ethylene glycol, formamide, hexane, methanol, 2-methoxyethanol, methyl butyl ketone, methylcyclohexane, N-methylpyrrolidone, nitromethane, pyridine, sulfolane, tetrahydrofuran, tetralin, toluene, 1,1,2-trichloroethene, and xylene.

In accordance with the disclosed subject matter, the excipient and therapeutic agent coating process can occur aseptically or be followed with terminal sterilization method such as E-beam, gamma irradiation, or ethylene oxide sterilization.

In accordance with the disclosed subject matter, excipients are utilized together with the therapeutic agent in the coating at ratios ranging from 1:20 to 20:1 excipient:drug by weight, preferably from 1:10 to 10:1, more preferably from 1:2 to 2:1. Preferably, the coating includes a plasticizer. In this regards, the excipient to plasticizer weight ratio is from about 20:1 to about 1:20, more preferably from 10:1 to 1:1.

In accordance with another embodiment of the disclosed subject matter, the coating includes various layers. In one embodiment, the coating includes first and second layers adsorbed to the surface of the balloon. The first layer typically consists of one therapeutic agent and one excipient and the second layer typically consists of a second therapeutic agent and second excipient. The drug coated balloon is designed such that the first and second layers each have a dissolution rate. Preferably, the dissolution profile of the first layer is different than the dissolution profile of the second layer. Providing layers with various dissolution profiles allows the coating to be tuned to an optimized range.

In accordance with yet another embodiment, the disclosed subject matter includes a method of increasing the efficiency of therapeutic transfer to a body lumen by implanting or inserting a medical device in a body lumen. The medical device includes an expandable member having an outer surface and a coating disposed on the outer surface of the medical device, the coating including a therapeutic agent and an excipient.

For example, and not limitation, the at least one therapeutic agent can include anti-proliferative, anti-inflammatory, antineoplastic, antiplatelet, anti-coagulant, anti-fibrin, antithrombotic, antimitotic, antibiotic, antiallergic and antioxidant compounds. Thus, the therapeutic agent can be, again without limitation, a synthetic inorganic or organic compound, a protein, a peptide, a polysaccharides and other sugars, a lipid, DNA and RNA nucleic acid sequences, an antisense oligonucleotide, an antibody, a receptor ligand, an enzyme, an adhesion peptide, a blood clot agent including streptokinase and tissue plasminogen activator, an antigen, a hormone, a growth factor, a ribozyme, and a retroviral vector. Preferably, however, the therapeutic agents include a cytostatic drug. The term “cytostatic” as used herein means a drug that mitigates cell proliferation, allows cell migration, and does not induce cell toxicity. These cytostatic drugs, include for the purpose of illustration and without limitation, macrolide antibiotics, rapamycin, everolimus, zotarolimus, biolimus, novolimus, myolimus, temsirolimus, deforolimus, structural derivatives and functional analogues of rapamycin, structural derivatives and functional analogues of everolimus, structural derivatives and functional analogues of zotarolimus and any macrolide immunosuppressive drugs. The term “antiproliferative” as used herein means a drug used to inhibit cell growth, such as chemotherapeutic drugs. Some non-limiting examples of antiproliferative drugs include taxanes, paclitaxel, and protaxel.

Therefore, in accordance with a preferred embodiment, a balloon for delivery of a cytostatic drug is provided. The outer surface of the balloon includes a tunable coating, the tunable coating including a first cytostatic drug and a first excipient and a second cytostatic drug and a second excipient. The first and second cytostatic drugs preferably have different dissolution rates during balloon inflation. The various dissolution rates allow for more effective and efficient delivery of the therapeutic agent.

With reference to the balloon construction, a polymeric expandable balloon material is preferred. For example, the polymeric material utilized to form the balloon body can be compliant, non-compliant or semi-compliant polymeric material or polymeric blends.

In one embodiment, the polymeric material is compliant such as but not limited to a polyamide/polyether block copolymer (commonly referred to as PEBA or polyether-block-amide). Preferably, the polyamide and polyether segments of the block copolymers can be linked through amide or ester linkages. The polyamide block can be selected from various aliphatic or aromatic polyamides known in the art. Preferably, the polyamide is aliphatic. Some non-limiting examples include nylon 12, nylon 11, nylon 9, nylon 6, nylon 6/12, nylon 6/11, nylon 6/9, and nylon 6/6. Preferably, the polyamide is nylon 12. The polyether block can be selected from various polyethers known in the art. Some non-limiting examples of polyether segments include poly(tetramethylene ether), tetramethylene ether, polyethylene glycol, polypropylene glycol, poly(pentamethylene ether) and poly(hexamethylene ether). Commercially available PEBA material can also be utilized such as for example, PEBAX® materials supplied by Arkema (France). Various techniques for forming a balloon from polyamide/polyether block copolymer is known in the art. One such example is disclosed in U.S. Pat. No. 6,406,457 to Wang, the disclosure of which is incorporated by reference.

In another embodiment, the balloon material is formed from polyamides. Preferably, the polyamide has substantial tensile strength, be resistant to pin-holing even after folding and unfolding, and be generally scratch resistant, such as those disclosed in U.S. Pat. No. 6,500,148 to Pinchuk, the disclosure of which is incorporated herein by reference. Some non-limiting examples of polyamide materials suitable for the balloon include nylon 12, nylon 11, nylon 9, nylon 69 and nylon 66. Preferably, the polyamide is nylon 12. Other suitable materials for constructing non-compliant balloons are polyesters such as polyethylene terephthalate) (PET), Hytrel thermoplastic polyester, and polyethylene.

In another embodiment, the balloon is formed of a polyurethane material, such as TECOTHANE® (Thermedics). TECOTHANE® is a thermoplastic, aromatic, polyether polyurethane synthesized from methylene disocyanate (MDI), polytetramethylene ether glycol (PTMEG) and 1,4 butanediol chain extender. TECOTHANE® grade 1065D is presently preferred, and has a Shore durometer of 65D, an elongation at break of about 300%, and a high tensile strength at yield of about 10,000 psi. However, other suitable grades can be used, including TECOTHANE® 1075D, having a Shore D hardness of 75. Other suitable compliant polymeric materials include ENGAGE® (DuPont Dow Elastomers (an ethylene alpha-olefin polymer) and EXACT® (Exxon Chemical), both of which are thermoplastic polymers. Other suitable compliant materials include, but are not limited to, elastomeric silicones, latexes, and urethanes.

The compliant material can be cross linked or uncrosslinked, depending upon the balloon material and characteristics required for a particular application. The presently preferred polyurethane balloon materials are not crosslinked. However, other suitable materials, such as the polyolefinic polymers ENGAGE® and EXACT®, are preferably crosslinked. By crosslinking the balloon compliant material, the final inflated balloon size can be controlled. Conventional crosslinking techniques can be used including thermal treatment and E-beam exposure. After crosslinking, initial pressurization, expansion, and preshrinking, the balloon will thereafter expand in a controlled manner to a reproducible diameter in response to a given inflation pressure, and thereby avoid overexpanding the stent (if used in a stent delivery system) to an undesirably large diameter.

In one embodiment, the balloon is formed from a low tensile set polymer such as a silicone-polyurethane copolymer. Preferably, the silicone-polyurethane is an ether urethane and more specifically an aliphatic ether urethane such as PURSIL AL 575A and PURSIL AL10, (Polymer Technology Group), and ELAST-EON 3-70A, (Elastomedics), which are silicone polyether urethane copolymers, and more specifically, aliphatic ether urethane cosiloxanes. In an alternative embodiment, the low tensile set polymer is a diene polymer. A variety of suitable diene polymers can be used such as but not limited to an isoprene such as an AB and ABA poly(styrene-block-isoprene), a neoprene, an AB and ABA poly(styrene-block-butadiene) such as styrene butadiene styrene (SBS) and styrene butadiene rubber (SBR), and 1,4-polybutadiene. Preferably, the diene polymer is an isoprene including isoprene copolymers and isoprene block copolymers such as poly(styrene-block-isoprene). A presently preferred isoprene is a styrene-isoprene-styrene block copolymer, such as Kraton 1161K available from Kraton, Inc. However, a variety of suitable isoprenes can be used including HT 200 available from Apex Medical, Kraton R 310 available from Kraton, and isoprene (i.e., 2-methyl-1,3-butadiene) available from Dupont Elastomers. Neoprene grades useful in the disclosed subject matter include HT 501 available from Apex Medical, and neoprene (i.e., polychloroprene) available from Dupont Elastomers, including Neoprene G, W, T and A types available from Dupont Elastomers.

In accordance with another aspect of the disclosed subject matter, the outer surface of the balloon is modified. In this regard, the balloon surface can include a textured surface, roughened surface, voids, spines, channels, dimples, pores, or microcapsules or a combination thereof, as will be described below.

In accordance with the disclosed subject matter, the balloon does not include a stent or is free of a stent. However, a stent can be mounted onto the coated balloon. The stent will not detrimentally affect coating integrity or drug delivery. The type of stent that can be used includes, but is not limited to, bare metal stent, balloon expandable stent, self expanding stent, drug eluting stent, prohealing stent, and self-expanding vulnerable plaque implant. The balloon can be coated independently of the stent or in conjunction with the stent coating process. The stent coating can contain the same or different therapeutic agents from the balloon catheter or expandable member. However, the particular coating on the balloon catheter or expandable member preferably has distinct release kinetics from the therapeutic coating on the stent.

In one embodiment of the disclosed subject matter, the balloon is formed of a porous elastomeric material having at least one void formed in the wall of the balloon surface. For example, the entire cross section of the balloon can contain a plurality of voids. Alternatively, the plurality of void can be distributed along select lengths of the balloon outer surface. For example and not limitation, the plurality of voids can be distributed only along only the working section of the balloon. The voids define an open space within the outer surface of the balloon. Preferably, the therapeutic agent is dispersed within the space defined by the plurality of voids across the cross section of the balloon outer surface.

In operation, the therapeutic agent is released or is expelled from the pores upon inflation of the balloon. In this regard, the durometer of the polymeric material of the balloon surface and in particular the depression of the void is sufficiently flexible to allow for expulsion of the therapeutic agent and/or coating contained within the plurality of voids upon inflation of the balloon. The expelled coating with therapeutic agent is released into the vessel lumen or into the tissue surrounding and contacting the inflated balloon.

In another embodiment, the balloon includes protrusions configured to contact or penetrate the arterial wall of a vessel upon inflation of the balloon. A coating containing therapeutic agent is disposed on the protrusions and when inflated the coating and/or therapeutic agent coats the tissue of the arterial wall. Alternatively, the balloon can include two concentric balloons in a nesting configuration. The coating with therapeutic agent is disposed between the two concentric balloons. Thus, the space between the two concentric balloons; one being an interior balloon and the other being an exterior balloon, acts as a reservoir. In this regard, the protrusions can include apertures for expulsion of the coating and/or therapeutic agent upon inflation of the interior and exterior concentric balloons. For example, as described in U.S. Pat. No. 6,991,617 to Hektner, the disclosure of which is incorporated herein by reference thereto. In another embodiment, the balloon can include longitudinal protrusions configured to form ridges on the balloon surface. As described in U.S. Pat. No. 7,273,417 to Wang, the entire disclosure of which is incorporated herein by reference, the ridges can be formed of filaments spaced equidistantly apart around the circumference of the balloon. However, a larger or smaller number of ridges can alternatively be used. The longitudinal ridges can be fully or partially enveloped by the polymeric material of the balloon.

In yet another embodiment of the disclosed subject matter, the balloon can include microcapsules on its outer surface. In this regard, the microcapsules are configured to encompass the coating and/or therapeutic agent. Upon inflation of the balloon the microcapsules located on the surface of the balloon contact the tissue of the arterial wall. Alternatively, the microcapsules can be formed in the wall of the balloon surface. The coating and/or therapeutic agent can be released from the microcapsules by fracturing of the microcapsules and/or diffusion from the microcapsule into the arterial wall. The microcapsules can be fabricated in accordance with the methods disclosed in U.S. Pat. No. 5,1023,402 to Dror or U.S. Pat. No. 6,129,705 to Grantz and the patents referenced therein, each of which is incorporated herein by reference in its entirety.

In accordance with another aspect of the disclosed subject matter, if desired, a protective sheath can be utilized to protect the coating from being rubbed off of the balloon during the movement of the coated balloon through the body lumen. The sheath is preferably made from an elastic and resilient material which conforms to the shape of the balloon and in particular is capable of expanding upon inflation of the balloon. The sheath preferably includes apertures along a length thereof. In operation, the inflation of the balloon causes the apertures of the sheath to widen for release of the coating and/or therapeutic agent to the tissue of the arterial wall. Preferably, the sheath has a thickness less than 10 mils. However, other thicknesses are possible.

In another embodiment, the sheath has at least one longitudinal line of weakness allowing the sheath to rupture upon inflation of the balloon and the release of the coating and/or therapeutic agent onto the tissue of the arterial wall of the vessel. Preferably, the sheath is formed from polymeric material known to be suitable for use in balloon catheters. Preferably, the sheath material is an elastomeric material which will also spring back when it splits to expose more of the body lumen to the coating. The line of weakness could be provided by various techniques known in the art. However, one non-limiting examples include perforating the sheath material. In operation, the sheath is placed over the coated balloon while in the deflated state. When the coated balloon is inflated, the sheath is expanded to the extent that it exceeds its elastic limit at the line of weakness and bursts to expose and therefore release the coating and/or therapeutic agent to the tissue of the arterial wall or vessel lumen. For example, see U.S. Pat. No. 5,370,614 to Amundson, the entire disclosure of which is incorporated by reference.

In accordance with another embodiment, an outer fibrous coating can be electrospun or stretched onto the medical device or balloon catheter to prevent drug loss during delivery. During balloon inflation, the coating is stretched and allows for coating solubilization and release. The fiber diameters and material properties can be fine tuned for optimal pore size and to release the particles containing the therapeutic agent. Fibrous coatings on expandable members are described in U.S. patent application Ser. No. 12/237,998 to R. von Oepen and U.S. patent application Ser. No. 12/238,026 to K. Ehrenreich, the disclosures of which are incorporated by reference in their entirety.

It is to be noted that the term “a” entity or “an” entity refers to one or more of that entity. For example, a protein refers to one or more proteins or at least one protein. As such, the terms “a”, “an”, “one or more”, and “at least one” can be used interchangeably herein. The terms “comprising,” “including,” and “having” can also be used interchangeably. In addition, the terms “amount” and “level” are also interchangeable and can be used to describe a concentration or a specific quantity. Furthermore, the term “selected from the group consisting of” refers to one or more members of the group in the list that follows, including mixtures (i.e. combinations) of two or more members.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to +/−20%, preferably up to +/−10%, more preferably up to +/−5%, and more preferably still up to +/−1% of a given value. Alternatively, particularly with respect to biological systems or processes, the term can mean within an order of magnitude, preferably within 5-fold, and more preferably within 2-fold, of a value. With reference to pharmaceutical compositions, the term “about” refers to a range that is acceptable for quality control standards of a product approved by regulatory authorities.

### EXAMPLES

The present application is further described by means of the examples, presented below. The use of such examples is illustrative only and in no way limits the scope and meaning of the disclosed subject matter or of any exemplified term.

### Example A

To simulate drug release from a drug coated balloon, a three step in-vitro release method was developed. This method consists of a sequential dip release in 37° C. porcine serum for 1 min, inflation of the balloon to nominal pressure (8 atm) in 37° C. porcine serum for I min and extraction release in 50% acetonitrile solution designed to mimic the balloon release during delivery to the lesion, drug delivery on inflation and the remaining drug on the balloon respectively. The resulting zotarolimus concentrations in the porcine serum supernatant are measured by liquid chromatography mass spectrometry (LCMS) and drug from the extraction measured by high performance liquid chromatography (HPLC).

This in-vitro release method was used to evaluate the drug release from zotarolimus (Zot):poly(vinylpyrrolidone) (PVP):glycerol drug coated balloons as a function of drug:excipient:plasticizer ratio (D:E:P) and PVP K-value. For the combined dip release and inflation release that simulates coating dissolution rate and drug delivery from a drug coated balloon, it is shown in FIG. 2 that a higher drug to excipient ratio such as D:E:P, 20:1:0.4 (w/w) resulted in a “soluble” coating dissolution rate with a dissolution time in the range of 1 min to 1 h releasing less than 5% of drug in 2 min. For lower D:E:P ratios and increasing amounts of plasticizer, the Zot:PVP:glycerol formulation demonstrated a “fast” dissolution rate less than 1 min releasing up to 90% of drug in 2 min. For a lower molecular weight or PVP K-value such as PVP C-15, the coating dissolution rate and drug release during the dip release was further increased to 30% as compared to the PVP C-30 coating at the same 1:1:0.4, D:E:P ratio which demonstrated less than 5% dip release.

### Example B

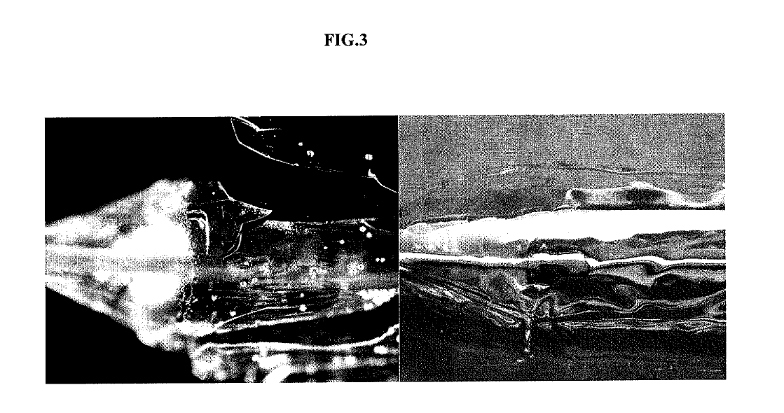

Without plasticizer a low MW PVP coating such as with a C-30 grade produces a glassy, brittle coating when dry (FIG. 3 left panel). With addition of glycerol plasticizer at 20 wt % the resulting coating was tough and pliable (FIG. 3 right panel). Zotarolimus:PVP:glycerol was coated onto Agiltrac PTA catheters at either 2:1:0.4 or 2:1:0.2 ratios by weight from acetone:ethanol 85:15 solvent. Post-coating the dried balloons were folded, pressed and sheathed. Drug recovery to target was measured by extracting the coated drug by HPLC._No significant difference was observed in mean drug recovery as an effect of fold, pressing and sheathing as shown in FIG. 3. This result indicated that brittle drug loss during dry catheter processing was minimal with at least a 2:1:0.2 ratio of drug:excipient:plasticizer.

### Example C

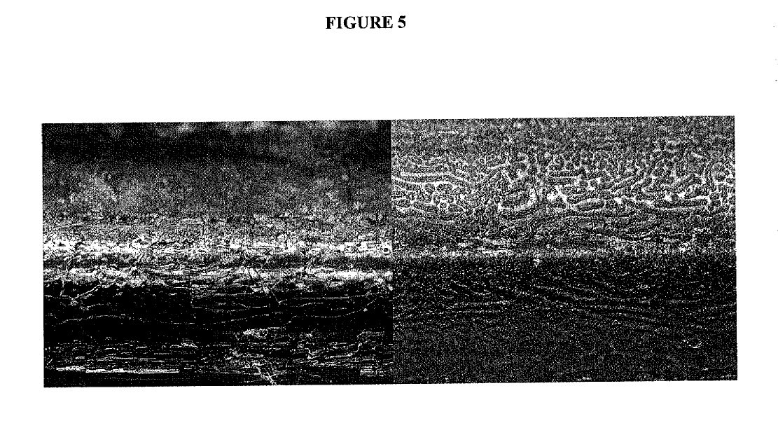

The resulting balloon coating morphology can be strongly influenced by the coating process conditions. For example, the morphology of a zotarolimus: PVP: glycerol 2:1:0.4 coating at 400 μg/cm² dose can be modified as a function of coating solvent and related evaporation rate. As shown in FIG. 5, coating from a faster evaporating 95:5 acetone:ethanol by weight solvent ratio produced white, glassy coating (left panel) while coating from a slower evaporating 85:15 acetone:ethanol by weight solvent ratio produced an opaque coating (right panel).

The disclosed subject matter can be embodied in other specific forms without departing from its spirit or essential characteristics. The described embodiments are to be considered in all respects only as illustrative and not restrictive. Thus, it is intended that the disclosed subject matter include modifications and variations that are within the scope of the appended claims and their equivalents. All references recited herein are incorporated herein in their entirety by specific reference.

What is claimed is:

1. A therapeutic coating formulation for coating a medical balloon comprising:
a cytostatic therapeutic agent;
an excipient; and
a plasticizer, wherein the weight ratio of the excipient to plasticizer is below 1 to 0.1.

2. The coating formulation of claim 1, wherein the balloon exhibits improved flexibility when dry.

3. The coating formulation of claim 1, wherein the excipient is PVP.

4. The coating formulation of claim 1, wherein the plasticizer is selected from the group consisting of glycerol and propylene glycol.

5. The coating of claim 1, wherein the excipient is PVP, and the plasticizer is glycerol.

6. The coating of claim 1, wherein the cytostatic therapeutic agent is selected from the group consisting of everolimus, zotarolimus, rapamycin, biolimus, myolimus, novolimus, deforolimus, temsirolimus, paclitaxel and protaxel.

7. The coating of claim 1, wherein the cytostatic therapeutic agent and excipient have a weight ratio of greater than 1:1.

8. The coating of claim 1, wherein the coating is applied to an outer surface of a medical balloon.

9. The coating of claim 8, wherein the coated medical balloon exhibits minimized drug loss during folding of the balloon.

10. The coating of claim 8, wherein the coated medical balloon exhibits improved drug recovery upon tracking the balloon through a lumen of a subject.

11. The coating formulation of claim 1, wherein the glass transition temperature of the hydrated coating is below 37° C.

12. The coating formulation of claim 1, wherein the formulation provides for enhanced tissue uptake of the therapeutic agent upon balloon inflation.
